(19) United States
(12) Patent Application Publication
(10) Pub. No.: US 2020/0353207 A1
(43) Pub. Date: Nov. 12, 2020

(54) Monolithic Self-Intermittent Catheterization System With Attached Extension

(71) Applicant: Gain Products, LLC, Fenton, MI (US)

(72) Inventors: William Michael Vickers, Holly, MI (US); Christine Marie McDunnough, Fenton, MI (US); Sharina Jones, Lake Orion, MI (US); Grover Jones, Lake Orion, MI (US)

(73) Assignee: Gain Products, LLC, Fenton, MI (US)

(21) Appl. No.: 16/405,961

(22) Filed: May 7, 2019

(51) Int. Cl. A61M 25/00; A61M 39/12

(52) U.S. Cl. CPC A61M 25/0017; A61M 25/0111; A61M 39/12

(57) ABSTRACT

A monolithic self-intermittent catheterization system contains a catheter fixed to a connector, and an extension tube that is fixed to the catheter. Accordingly, the catheter, connector, and the drainage tube all fluidly communicate with each other. A quick release retainer may be used to retain the extension tube in a compact state, whereby a user can readily release the extension tube for ease of use. Yet further, the extension tube is made from a non-resilient material and has no shape memory, thereby also enhancing the ease and safety of use.

### BACKGROUND OF THE INVENTION

Catheters have been developed for some time, with improvements periodically being made in the art. One challenge that remains is the usefulness or functionality of the various catheters to those users with limited dexterity, limited mobility, and/or limited vision, perhaps. Another ongoing challenge is to limit or mitigate bacterial contamination during the catheterization process in order to reduce the risk of urinary tract infections, which can plague the self-intermittent catheter user and may result in severe health concerns including septicemia and death.

Self-intermittent catheterization systems are typically used by anyone with a Neurogenic Bladder due to Spinal Cord Injury, Multiple Sclerosis, Stroke, or any other number of maladies or traumas which leaves the person with difficulty or full inability to pass urine unaided. Daily use of self-intermittent catheters is a way of life for those with Neurogenic Bladder. For example, home use, hospital use, and use in public restrooms of these types of catheters may occur between six to eight times every day, whereby the user uses each catheter one time and then typically disposes of the same. These types of catheterization systems typically contain a catheter for insertion through the urethra and into the bladder and a connector or handle for handling the catheter without actually touching the insertion portion of the catheter. The catheter may or may not be pre-lubricated as desired.

When using a self-intermittent catheter, an extension tube may be purchased or provided as an auxiliary complement to the catheter. If desired, an extension tube may be connected to the connector such that the urine or fluid passing through the catheter may also pass through the extension tube into the evacuation vessel such as a toilet, for example. This facilitates ease of use during catheterization in public restrooms or scenarios where accessibility may be problematic, and for many other issues where restroom proximity is an issue.

One disadvantage with this approach is that the user must attach the extension tube to the connector. When the user has limited dexterity, this presents a challenge. For example, connecting the auxiliary extension tube to the connector is difficult or virtually impossible for those having upper extremity impairment, or limited hand or finger dexterity. Yet further, the addition of this extra step in the process increases handling and exposure of the catheter itself and may increase the likelihood of the insertion portion of the catheter coming into contact with bacteria. Even further, if the user drops the catheter or the extension tube, the hygienic field necessary to protect the user from infection could potentially be hampered, particularly if a pre-lubricated catheter (for example) slips from the user's hand and is then retrieved for use anyway. Yet further, because of the expense of the extension tubes, and because insurance companies may not for example provide more than two extension tubes per month, the extension tubes are typically re-used by rinsing and cleaning the extension tubes before re-use. Reusing the extension tubes therefore may present a risk of introducing bacteria which may lead to subsequent urinary tract infections.

Yet further, because of limited dexterity and/or mobility, it is oftentimes necessary for quadriplegics and other similarly-situated users, to employ or otherwise provide an assistant that can aid them in inserting an associated self-intermittent catheter for use. This, of course, adversely affects the independence and autonomy of the user, and increases the need for care-giving costs, resources, and/or time. In particular, a known self-intermittent catheterization system may contain a catheter connected to a relatively smaller connecter wherein the catheter may, for example, be pre-lubricated when packaged for use. As a result, the user may find it challenging not only to maintain the cleanliness or hygienic field when inserting the catheter for use, but again, may sometimes drop the catheter because of the slippery nature of the device.

When a user elects to use a catheter that has dropped on the ground or floor, an infection risk may ensue. For example, urinary tract bacterial infections are not uncommon for home users of catheters. Accordingly, serious illness or injury could possibly result from the use of contaminated catheters.

In the same realm, there is another type of urinary drainage system typically used by bed-ridden or institutionalized patients which is an indwelling catheterization system. Indwelling catheterization systems are long term or semi-permanent devices designed to remain stationed in the bladder and urethra for the purpose of being open for continual drainage of urine into a bag. It has been found that this type of prolonged use may also increase the likelihood of contamination. These types of catheterization systems are meant to be inserted into the patient, and then used for a time while in the hospital or care-giving facility. Patients may also use indwelling systems in a home setting on a permanent or semi-permanent basis. Indwelling catheters typically contain a catheter for insertion into the urinary system of the user, a balloon for inflating within the bladder, a connecter or handle for handling the catheter without touching it, an attachment tube connected to the catheter by and/or through the connector for draining the urine, and a collection vessel such as a bag connected to the drainage tube for collection of urine, for example. If possible, it is preferred that users of catheters use self-intermittent catheters rather than indwelling catheters because of the relative greater safety of self-intermittent catheters.

In sum, because of the potential and inherent known risks of using these catheters, improvements directed to minimizing the occurrences of infections are in demand, and would be an improvement in the art.

### SUMMARY OF THE INVENTION

A novel monolithic self-intermittent catheterization system is presented. A monolithic self-intermittent catheterization system contains a catheter fixed to a connector or handle with an attached extension. The extension or evacuation tube is also fixed to the connecter and fluidly communicates with the catheter. Importantly, the extension tube is fixed to the connector just as the catheter is, thereby immediately affecting fluid communication from the catheter through the connector and into the extension tube, upon insertion of the catheter within a user's urinary tract.

If desired, the catheter may be pre-lubricated. In yet another novel aspect of the present invention, the associated packaging contains a wet chamber and a dry chamber, isolated from the fluid or wetness within the wet chamber. In accordance with the present invention, therefore, the catheter portion of the system is thereby packaged in a fluidized chamber of the package, and the connector/handle and the attached extension tube are contained in a dry chamber within the same packaging. Accordingly, whether the catheter portion is pre-lubricated or not, the user has a dry connector and a dry drainage tube, thereby providing the user with a greater gripping area to assist in inserting the catheter portion for use, and reducing the likelihood of contaminating the catheter either by direct touch, or by dropping the catheter while attempting to insert it.

In yet another aspect of the invention, the extension and evacuation tube, hereinafter “extension tube”, is made from a non-memory polymeric or other suitable material. As such, when the extension tube is coiled for efficiency of packaging, it will readily discard its coiled shape once it is removed from the package. Yet further, and in accordance with another aspect of the present invention, the packaged extension tube is retained in compact state such as a coiled or folded state, by a quick-release retainer. For example, the retainer may be a paper tie adhesively fixed about the coiled or folded extension tube, wherein the retainer contains a tab that may be readily pulled by the fingers, mouth, or teeth of the user, to thereby release the extension tube from its packaged state. In this way, the self-intermittent catheterization system may be readily removed from the packaging and arranged for use by the user.

In sum, the present system provides a monolithic self-intermittent catheterization system having a self-intermittent catheter with a fixed extension tube.

### BRIEF DESCRIPTION OF THE DRAWINGS

FIG. 1 is a perspective view of an exemplary monolithic self-intermittent catheterization system, in an unreleased state.

FIG. 2 is a side view of the embodiment shown in FIG. 1.

FIG. 3 is a side view of a second exemplary embodiment of a monolithic self-intermittent catheterization system, again in an unreleased state.

FIG. 4 is a perspective view of an exemplary connector in accordance with the present invention.

FIG. 5 is a view of the embodiment of FIG. 1 in a released state.

FIG. 6 is a schematic view of the embodiment of FIG. 1 in an exemplary package.

### DETAILED DESCRIPTION OF THE INVENTION

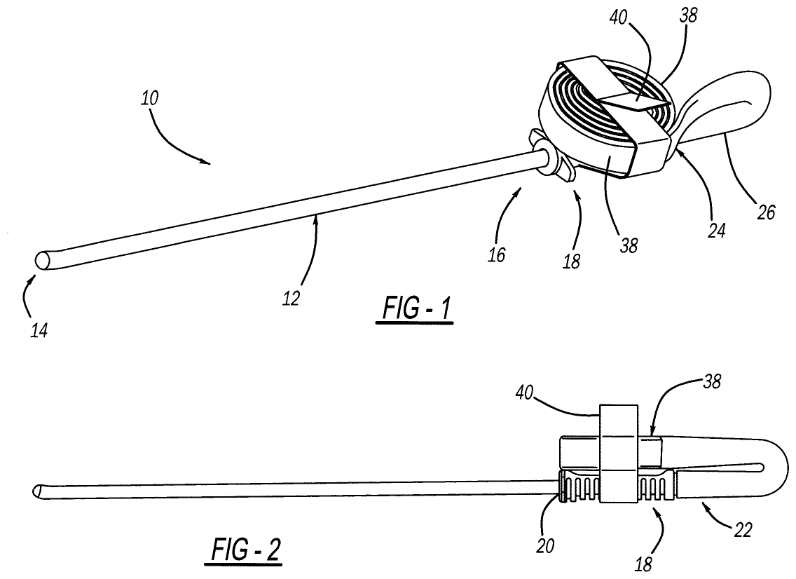

As shown in FIG. 1, a novel monolithic self-intermittent catheterization system 10 is provided. The term “monolithic” indicates that the system components identified herein are provided or integrated within a single assembly, thereby enhancing the safety and simplifying the use of the self-intermittent catheterization system for the user. The components can be fixed in a known manner, or, the components can be chemically welded, such as with a cross-linking and/or solvent processing, for example. A catheter 12, formed in a known manner, has a first open end 14 for insertion into a urinary tract, and a second open end 16. A handle or connector 18 contains a third open end 20 and a fourth open end 22, wherein the third open end 20 is connected to the second end 16, thereby affecting fluid communication between the catheter 12 and the connector 18, for drainage of an associated urinary tract. The catheter 12 may be pre-lubricated as packaged, or, the catheter 12 may be lubricated after it is removed from its packaging.

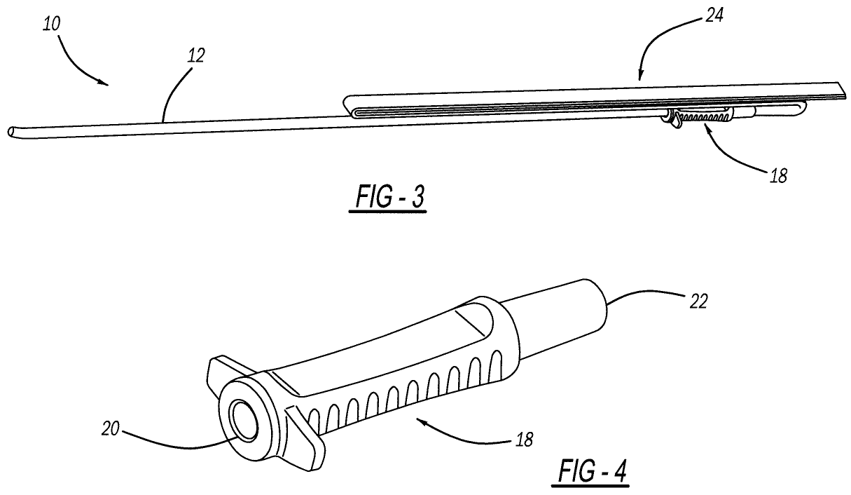

In accordance with the present invention, an extension tube 24 contains a fifth open end 26 and sixth open end 28, wherein the fifth open end 26 is fixed to the fourth open end 22, thereby affecting fluid communication with the connector 18 and the catheter 12. As shown in FIGS. 1-3, the extension tube 24 may be coiled or folded upon itself, or provided in a compact state, to thereby provide a packaging advantage by reducing the size and amount of packaging materials, and by also facilitating the separate dry storage of the extension tube 24 within the associated packaging.

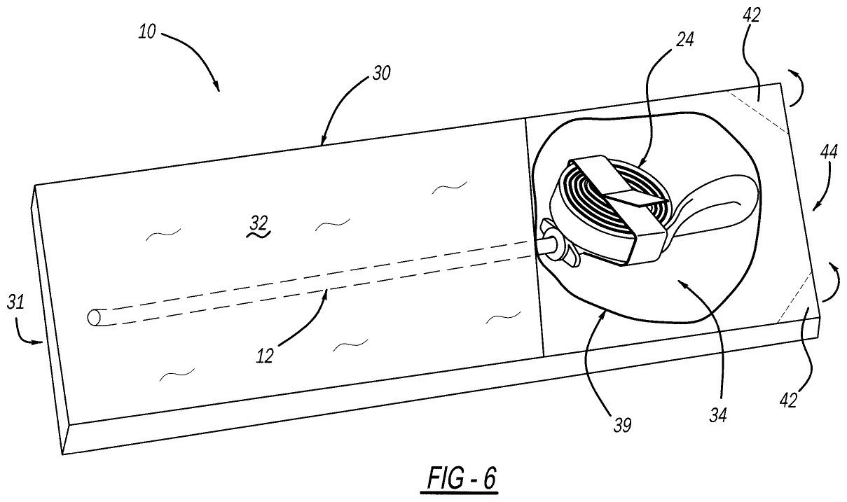

FIG. 6 illustrates an exemplary package 30 for a monolithic catheterization system of the present invention. As shown, a housing 31 contains a wet portion or chamber 32 for maintaining a lubricated catheter 12. As also shown, a dry portion or chamber 34 is juxtaposed to the wet chamber 32, and may for example, be a dry area 34 formed by a polymeric material or bag 36 surrounding the connector 18 and the coiled or folded extension tube 24. If desired, the package 30 may be formed with one or more quick-release tabs 42 that are designed to easily pull away a housing cover 44 from the enclosed package 30 to provide access to the catheterization system 10, thereby addressing the need of those with limited dexterity.

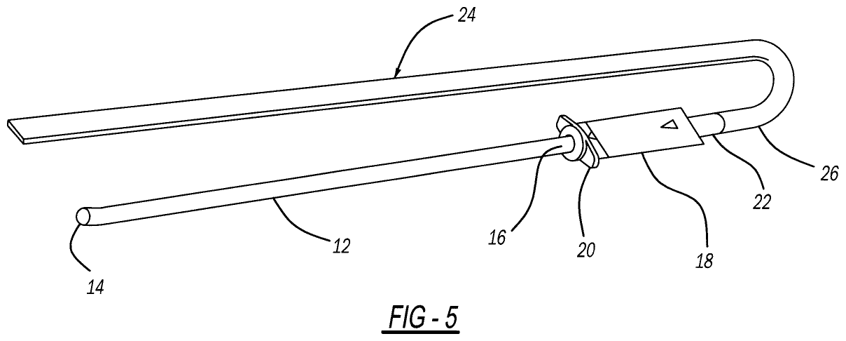

In yet another aspect of the present invention, the compact, coiled, or folded extension tube 24 may be formed from a material that when uncoiled or unfolded, does not retain a memory of its former coiled or folded state, and thereby relaxes in its released state. Stated another way, the material may be a polymeric or other suitable material that exhibits a non-memory or non-resilient release from its folded or coiled packaging configuration. The term “non-resilient” as used herein indicates that the polymer does not return to its coiled, folded, or packaged state once it is released from the package. The term “non-memory” as used herein indicates that the polymer does not return either wholly or partially, to its compact state. Exemplary materials used to form the drainage tube 24 and the connector 18 may include, but are not limited to, extruded unreinforced silicone, thermoplastic polyurethane (TPU), and thermoplastic elastomers (TPE). FIG. 5 illustrates how the drainage tube 24 of the embodiment of FIG. 1 is preferably relaxed when released from its packaged or coiled state.

Yet further, and in accordance with another aspect of the present invention, the packaged extension tube 24 is retained in a coiled or folded state by a quick-release retainer 38. For example, the retainer 38 may be tear-away tape, or a paper band or tie adhesively fixed about the coiled or folded extension tube 24, wherein the retainer 38 contains a tab 40 that may be readily pulled by the fingers, mouth, or teeth of the user, to thereby release the drainage tube 24 from its packaged state. In this way, the self-intermittent catheterization system 10 may be readily removed from the packaging 30 and arranged for use by the user.

The components of the present invention may be made from polymers as indicated herein, and may be formed by known processes. Other suitable materials may also be used so long as they are suitable in the catheter art and so long as they comply with the features as indicated herein, so long as the drainage tube 24 is flexible and “non-resilient” for example. U.S. Pat. Nos. 4,194,509, 7,537,589, 9,731,093, 9,821,139, and 10,207,043 exemplify related art and are incorporated by reference as if fully stated.

It will be understood that the foregoing descriptions of various embodiments of the present invention is for illustrative purposes only. As such, the various structural and operational features herein disclosed are susceptible to a number of modifications, none of which departs from the scope of the present invention as described above, shown in the drawings, and as stated in the appended claims.

What is claimed is:

1. A self-intermittent catheterization system comprising:
a catheter having a first open end and a second open end;
a connector having a third open end and a fourth open end, said third open end fixed to said second open end to provide fluid communication between the catheter and the connector; and
an extension tube having a fifth open end and a sixth open end, said fifth open end fixed to said fourth open end to provide fluid communication between the connector and the extension tube,
wherein said self-intermittent catheterization system is a monolithic system.

2. The system of claim 1 wherein said extension tube is retained in a compact state, and wherein said system further comprises a quick-release restraint to retain said extension tube in said compact state.

3. The system of claim 2 wherein said extension tube is retained in a coiled or folded state.

4. The system of claim 1 wherein said extension tube is non-resilient.

5. The system of claim 1 wherein said extension tube has no shape-memory.

6. The system of claim 2 wherein said quick-release restraint is a tear-away strap.

7. The system of claim 2 wherein said quick-release restraint is a paper band and tab adhesively fixed about said extension tube.

8. A self-intermittent catheterization system comprising:
a catheter having a first open end and a second open end;
a connector having a third open end and a fourth open end, said third open end fixed to said second open end to provide fluid communication between the catheter and the connector; and
an extension tube having a fifth open end and a sixth open end, said fifth open end fixed to said fourth open end to provide fluid communication between the connector and the extension tube,
wherein said self-intermittent catheterization system is a monolithic system, and, said extension tube is retained and configured in a compact state prior to use.

9. The system of claim 8 wherein said extension tube is retained in a coiled or folded state.

10. The system of claim 8 further comprising a quick-release retainer strapped about the compact extension tube.

11. The system of claim 10 wherein said quick-release retainer is a tear-away strap.

12. The system of claim 10 wherein said quick-release retainer is a paper band and tab fixed about said extension tube.

13. A self-intermittent catheterization system comprising:
a catheter having a first open end and a second open end;
a connector having a third open end and a fourth open end, said third open end fixed to said second open end to provide fluid communication between the catheter and the connector;
an extension tube having a fifth open end and a sixth open end, said fifth open end fixed to said fourth open end to provide fluid communication between the connector and the extension tube, and
a system package containing a wet portion for lubricating said catheter, and, a dry portion for housing the connecter and extension tube,
wherein said self-intermittent catheterization system is a monolithic system, and, said extension tube is retained and configured in a compact state.

14. The system of claim 13 wherein said extension tube is retained in a coiled or folded state.

15. The system of claim 13 further comprising a quick-release retainer strapped about the compact extension tube.

16. The system of claim 15 wherein said quick-release retainer is a tear-away strap.

17. The system of claim 15 wherein said quick-release retainer is a paper band and tab fixed about said extension tube.

18. The system of claim 15 wherein said quick-release retainer is formed with a quick-release tab to affect ease of release.

19. The system of claim 15 wherein said package has one or more tabs at one or more respective outer areas of the package, for ease of opening said package.

20. The system of claim 15 wherein said wet portion is a wet chamber, and, said dry portion is a dry chamber formed from a plastic material.
